(19) United States
(12) Patent Application Publication
(10) Pub. No.: US 2016/0002127 A1
(43) Pub. Date: Jan. 7, 2016

(54) PROCESS FOR THE PRODUCTION OF CHLORINATED PROPANES

(71) Applicant: BLUE CUBE IP LLC, Midland, MI (US)

(72) Inventors: Max M. TIRTOWIDJOJO, Lake Jackson, TX (US); David S. LAITAR, Midland, MI (US)

(73) Assignee: Blue Cube IP LLC, Midland, MI (US)

(21) Appl. No.: 14/771,215

(22) PCT Filed: Feb. 28, 2014
(86) PCT No.: PCT/US14/19211

§ 371 Date: Aug. 28, 2015

Related U.S. Application Data
(60) Provisional application No. 61/770,747, filed on Feb. 28, 2013.

(51) Int. Cl. C07C 17/10; C07C 17/25; C07C 17/275

(52) U.S. Cl. CPC C07C 17/10; C07C 17/275; C07C 17/25

(57) ABSTRACT

Processes for the production of chlorinated propanes are provided. The processes comprise catalyzing the chlorination of 1,1,1,3-tetrachloropropane with aluminum chloride, either alone or in combination with ferric chloride. Low intensity conditions are appropriate for the process, e.g., temperatures of from ambient to 100° C. and pressures of from ambient to 200 psig may be used. Even though low intensity conditions are used, the aluminum chloride provides at least 1.5 times greater the conversion rate and/or productivity of 1,1,1,3-tetrachloropropane as compared to ferric chloride when used as a single catalyst under similar processing conditions.

### FIELD

The present invention relates to processes for the production of chlorinated propanes.

### BACKGROUND

Hydrofluorocarbon (HFC) products are widely utilized in many applications, including refrigeration, air conditioning, foam expansion, and as propellants for aerosol products including medical aerosol devices. Although HFC's have proven to be more climate friendly than the chlorofluorocarbon and hydrochlorofluorocarbon products that they replaced, it has now been discovered that they exhibit an appreciable global warming potential (GWP).

The search for more acceptable alternatives to current fluorocarbon products has led to the emergence of hydrofluoroolefin (HFO) products. Relative to their predecessors, HFOs are expected to exert less impact on the atmosphere in the form of a lesser, or no, detrimental impact on the ozone layer and their lower GWP as compared to HFC's. Advantageously, HFO's also exhibit low flammability and low toxicity.

As the environmental, and thus, economic importance of HFO's has developed, so has the demand for precursors utilized in their production. Many desirable HFO compounds, e.g., such as 2,3,3,3-tetrafluoroprop-1-ene or 1,3,3,3-tetrafluoroprop-1-ene, may typically be produced utilizing feedstocks of chlorocarbons, and in particular, highly chlorinated propanes, e.g., pentachloropropanes.

Unfortunately, these pentachloropropanes have proven difficult to manufacture using acceptable process conditions and in commercially acceptable regioselectivities and yields. For example, conventional processes for the production of 1,1,1,2,3-pentachloropropane (such as those disclosed in U.S. Pat. No. 8,115,038) can provide acceptable selectivity to the desired pentachloropropane isomer, but only after extended reaction times that can render this process suboptimal for commercial applications.

It would thus be desirable to provide improved processes for the production of chlorocarbon precursors useful as feedstocks in the synthesis of refrigerants and other commercial products. More particularly, such processes would provide an improvement over the current state of the art if they provided similar, or greater, regioselectivity to the desired product in lessor reaction time relative to conventional methods.

### BRIEF DESCRIPTION

The present invention provides efficient processes for the production of chlorinated propanes. The process makes use of a single catalyst, aluminum chloride, other than ferric chloride. It has now been surprisingly discovered that aluminum chloride provides as great, or better, conversions of the starting material and/or selectivity to the desired product, in a shorter amount of time than ferric chloride. Time savings are thus provided. Mild reaction conditions are also used, and utility cost savings may also be seen.

In one aspect, a process for the production of 1,1,1,3-pentachloropropane from 1,1,1,3-tetrachloropropane is provided. The process comprises catalyzing the chlorination of 1,1,1,3-tetrachloropropane with aluminum chloride, either alone or in combination with ferric chloride. The process may be carried out in the presence of a solvent, and suitable solvents include carbon tetrachloride, sulfuryl chloride, one or more tetrachloropropanes, one or more pentachloropropanes, one or more hexachloropropanes, or a combination of any number of these.

Low intensity conditions are appropriate for the process, e.g., temperatures of from ambient to 100° C. and pressures of from ambient to 200 psig may be used. Even so, the aluminum chloride provides at least 1.5, or two times, or three times, or four times, or five times, or even six times or greater, the conversion rate and/or productivity of 1,1,1,3-tetrachloropropane as compared to ferric chloride when used as a single catalyst under similar processing conditions. In some embodiments, for example, at least a 80%, or even 100%, conversion of the 1,1,1,3-tetrachloropropane is provided with productivity of greater than 360 gr/L/min. In these, and other, embodiments, a yield of 1,1,1,2,3-pentachloropropane of at least 75%. or at least 90%, can be provided, with a productivity of greater than 360 gr/L/min. Productivities of at least 1000 gr/hr/L may also be seen.

The 1,1,1,3-tetrachloropropane may be prepared in situ, e.g., via the reaction of ethylene and carbon tetrachloride. This reaction may be catalyzed, in which case, Lewis acid catalysts, including ferric chloride, aluminum chloride, iodine, titanium chloride, antimony pentachloride, boron trichloride, one or more lanthanum halides, one or more metal triflates, or combinations of these, are suitable, although in this reaction, ferric chloride provides sufficient selectivity for commercial production and can be used alone.

In some embodiments, the 1,1,1,2,3-pentachloropropane produced by the process may be dehydrochlorinated to provide 1,1,2,3-tetrachloropropene. This dehydrochlorination may either be conducted catalytically or using caustic. If conducted catalytically, Lewis acid catalysts are again suitable.

### DESCRIPTION OF THE FIGURES

FIG. 1 shows a schematic representation of a process according to one embodiment.

### DETAILED DESCRIPTION

The present specification provides certain definitions and methods to better define the present invention and to guide those of ordinary skill in the art in the practice of the present invention. Provision, or lack of the provision, of a definition for a particular term or phrase is not meant to imply any particular importance, or lack thereof. Rather, and unless otherwise noted, terms are to be understood according to conventional usage by those of ordinary skill in the relevant art.

The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced item, and the terms “front”, “back”, “bottom”, and/or “top”, unless otherwise noted, are merely used for convenience of description, and are not limited to any one position or spatial orientation.

If ranges are disclosed, the endpoints of all ranges directed to the same component or property are inclusive and independently combinable (e.g., ranges of “up to 25 wt. %, or, more specifically, 5 wt. % to 20 wt. %,” is inclusive of the endpoints and all intermediate values of the ranges of “5 wt. % to 25 wt. %,” etc.). As used herein, percent (%) conversion is meant to indicate change in molar or mass flow of reactant in a reactor in ratio to the incoming flow, while percent (%) selectivity means the change in molar flow rate of product in a reactor in ratio to the change of molar flow rate of a reactant.

Reference throughout the specification to “one embodiment” or “an embodiment” means that a particular feature, structure, or characteristic described in connection with an embodiment is included in at least one embodiment. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

As used herein, the phrase “conversion rate” is meant to indicate conversion per unit time. The term “productivity” is meant to indicate product produced in weight or moles divided by both unit of time (hour) and reactor volume (cm³).

The present invention provides efficient processes for the production of chlorinated propanes. More particularly, in the present process, 1,1,1,3-tetrachloropropane is chlorinated in the presence of aluminum chloride to provide 1,1,1,2,3-pentachloropropane. The aluminum chloride may be used alone, or in combination with ferric chloride, although ferric chloride used alone is not within the scope of the present invention, since it catalyzes the chlorination of 1,1,1,3-tetrachloropropane inefficiently.

More particularly, although ferric chloride can provide a conversion rate of 1,1,1,3-tetrachloride of almost 500%/hr, aluminum chloride can provide conversion rates of greater than 500%/hr, or greater than 600%/hr, or greater than 750%/hr, or greater than 1000%/hr, or greater than 1500%/hr, or greater than 2000%/hr, or greater than 2500%/hr, or greater than 3000%/hr, or even greater than 3300%/hr. Also, whereas ferric chloride can provide productivities of almost 350%, aluminum chloride can provide productivities of greater than 350%, or greater than 400%, or greater than 500%, or greater than 600%, or greater than 700%, or greater than 800%, or greater than 900%, or greater than 1000%, or greater than 1500%, or even greater than 2000% of 1,1,1,2,3-pentachloropropane from 1,1,1,3-tetrachloropropane.

Stated another way, aluminum chloride can provide at least 1.5 times, two times, three times, four times, five times, or even greater than six times the productivity of 1,1,1,3-tetrachloropropane than ferric chloride, under the same processing conditions.

It has now been surprisingly discovered that, although aluminum chloride has been used as a component of a multicatalyst system for the chlorination of alkanes, they have not been used alone in such reactions. Aluminum chloride in particular, has conventionally been utilized with at least one other catalyst, oftentimes iodine. In contrast, the present inventors have now discovered that aluminum chloride may be used as an ionic chlorination catalyst, and acts to transform 1,1,1,2-tetrachloropropane with high conversion rates and/or productivity to 1,1 1,2,3-pentachloropropane.

In some embodiments, for example, at least a 80%, or even 100%, conversion of the 1,1,1,3-tetrachloropropane is provided with productivity of greater than 360 gr/L/min. In these, and other, embodiments, a yield of 1,1,1,2,3-pentachloropropane of at least 75%, or at least 90%, can be provided, with a productivity of greater than 360 gr/L/min. Productivities of at least 1000 gr/hr/L may also be seen.

These are unexpected and surprising results given that the starting material, 1,1,1,3-tetrachloropropane, does not have a chlorine on the second carbon atom. That is, although stronger Lewis acid catalysts than FeCl₃ such as AlCl₃ have been shown to be effective at catalyzing the chlorination of chlorinated alkanes, such as 1,2-dichloropropane, to produce more highly chlorinated alkanes, the addition of chlorine atoms has only been shown to occur preferentially on carbons already having a chlorine atom(s) attached thereto. In this instance, chlorine is added to a previously nonchlorinated carbon, to provide the desired end product, 1,1,1,2,3-pentachloropropane at the surprising yields and productivities disclosed herein.

Improvement over the excellent conversion rates and productivities provided by the use of aluminum chloride alone may be difficult to imagine, however, incremental improvement is possible. Since the use of ferric chloride in combination with aluminum chloride is not expected to detrimentally impact the process, it may be used in such combination if convenient and/or otherwise desirable to do so, in which case the aforementioned incremental improvements may be seen.

The chlorination of 1,1,1,3-tetrachloropropane may be carried out using a chlorination agent, and several of these are known in the art. For example, suitable chlorination agents include, but are not limited to chlorine, and/or sulfuryl chloride (SO₂Cl₂). Combinations of chlorinating agents may also be used. Either or both Cl₂ and sulfuryl chloride may be particularly effective when aided by the use of the aforementioned Lewis acid catalysts, although sulfuryl chloride may offer the benefit of also acting as a solvent for the process, should the same be desired.

In some embodiments, the 1,1,1,2,3-pentachloropopane may be dehydrochlorinated to provide 1,1,2,3-tetrachloropropene, and in such embodiments, the advantages provided by the process, e.g., via the excellent conversion of 1,1,1,3-tetrachloropropane and yield to 1,1,1,2,3-pentachloropropane are expected to carry forward so that similarly advantageous yields of 1,1,2,3-tetrachloropropene are seen.

The dehydrochlorination of 1,1,1,2,3-pentachloropropane may be carried out in the liquid or gas phase, either in the presence of, or without, catalyst. Catalytic dehydrochlorination provides the advantage of reducing the use of liquid caustic, and also provides the potential to recover anhydrous HCl from the process, which is a higher value byproduct than aqueous HCl. If the use of catalysts is desired, suitable dehydrochlorination catalysts include, but are not limited to, any of the Lewis acid catalysts mentioned above, as well as ferric chloride, as a substitute to caustic.

In other embodiments, the dehydrochlorination of 1,1,1,2,3-pentachloropropane may be conducted in the presence of a liquid caustic. Although vapor phase or solution-phase Lewis acid catalyzed dehydrochlorinations advantageously result in the formation of a higher value byproduct than caustic mediated dehydrochlorinations, caustic mediated dehydrochlorination reactions can provide cost savings since evaporation of reactants is not required. The lower reaction temperatures used in liquid phase reactions may also result in lower fouling rates than the higher temperatures used in connection with gas phase reactions, and so reactor lifetimes may also be optimized when a liquid phase dehydrochlorination is utilized.

Many chemical bases are known in the art to be useful for liquid dehydrochlorinations, and any of these can be used. For example, suitable bases include, but are not limited to, alkali metal hydroxides, such as sodium hydroxide, potassium hydroxide, calcium hydroxide; alkali metal carbonates such as sodium carbonate; lithium, rubidium, and cesium or combinations of these. Phase transfer catalysts such as quaternary ammonium and quaternary phosphonium salts (e.g. benzyltrimethylammonium chloride or hexadecyltributylphosphonium bromide) can also be added to improve the dehydrochlorination reaction rate with these chemical bases.

Any or all of the catalysts utilized in the process can be provided either in bulk or in connection with a substrate, such as activated carbon, graphite, silica, alumina, zeolites, fluorinated graphite and fluorinated alumina. Whatever the desired catalyst(s), or format thereof, those of ordinary skill in the art are well aware of methods of determining the appropriate format and method of introduction thereof. For example, many catalysts are typically introduced into the reactor zone as a separate feed, or in solution with other reactants.

The amount of the aluminum chloride and dehydrochlorination catalyst (if any) utilized will depend upon the particular catalyst chosen as well as the other reaction conditions. Generally speaking, in those embodiments of the invention wherein the utilization of a catalyst is desired, enough of the catalyst should be utilized to provide some improvement to reaction process conditions (e.g., a reduction in required temperature) or realized products, but yet not be more than will provide any additional benefit, if only for reasons of economic practicality.

For purposes of illustration only then, it is expected, that useful concentrations of aluminum chloride will range from 0.001% to 20% by weight, or from 0.01% to 10%, or from 0.1% to 5 wt. %, inclusive of all subranges therebetween. If a dehydrochlorination catalyst is utilized for the dehydrochlorination step, useful concentrations may range from 0.01 wt. % to 5 wt. %, or from 0.05 wt. % to 2 wt. % at temperatures of from 70° C. to 200° C. If a phase transfer catalyst is utilized, useful amounts may be 0.1 wt % or less. If a chemical base is utilized for the dehydrochlorination, useful concentrations of these will range from 0.01 to 20 grmole/L, or from 0.1 grmole/L to 15 grmole/L, or from 1 grmole/L to 10 grmole/L, inclusive of all subranges therebetween. Relative concentrations of each catalyst/base are given relative to the feed, e.g., 1,1,1,3-tetrachloropropane or 1,1,1,2,3-pentachloropropane as the case may be.

The reaction conditions under which the process is carried out are advantageously low intensity. That is, low temperatures, e.g., of less than 100° C., or less than 90° C., or less than 80° C. or less than 70° C., or less than 60° C., or less than 50° C., or even as low as 40° C. may be utilized and the desired selectivities to the tri-, tetra-, and/or pentachloroalkanes yet be realized, in some embodiments, temperatures of from ambient to 100° C. or from 40° C. to 70° C. or 55° C. to 65° C. may be utilized. Similarly, ambient pressure is suitable for carrying out the process, or pressures within 200, or 150, or 100, or 50, or 40, or 30, or 20, or even 10 psig, of ambient are suitable.

Reactor occupancy is significantly improved relative to conventional processes for the production of 1,1,1,2,3-pentachloropropane from 1,1,1,3-tetrachloropropane—for example, reactor occupancy times of less than 10 minutes, or less than 5 minutes, or less than 2 minutes, or less than 1 minutes, or less than 0.5 minutes are possible. The reactor may be any suitable liquid phase reactor, such as a batch or continuous stirred tank autoclave reactor with an internal cooling coil. A shell and multitube exchanger followed by vapor liquid disengagement tank or vessel can also be used.

In additional embodiments, one or more reaction conditions of the process may be optimized, in order to provide even further advantages, i.e., improvements in selectivity, conversion or production of reaction by-products. In certain embodiments, multiple reaction conditions are optimized and even further improvements in selectivity, conversion and production of reaction by-products produced can be seen.

Reaction conditions of the process that may be optimized include any reaction condition conveniently adjusted, e.g., that may be adjusted via utilization of equipment and/or materials already present in the manufacturing footprint, or that may be obtained at low resource cost. Examples of such conditions may include, but are not limited to, adjustments to temperature, pressure, flow rates, molar ratios of reactants, etc.

That being said, the particular conditions employed at each step described herein are not critical, and are readily determined by those of ordinary skill in the art. What is important is that a feedstream comprising 1,1,1,3-tetrachloropropane is chlorinated to provide 1,1,1,2,3-pentachloropropane using aluminum chloride, either alone, or in combination with ferric chloride. Those of ordinary skill in the art will readily be able to determine suitable equipment for each step, as well as the particular conditions at which the chlorination, dehydrochlorination, separation, drying, and isomerization steps may be conducted.

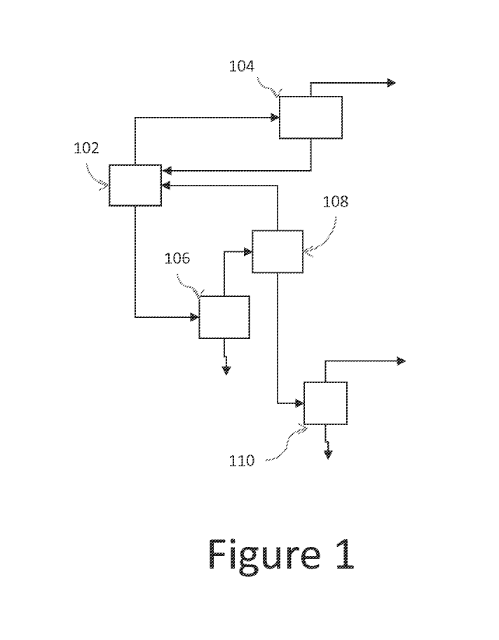

A schematic illustration of one embodiment of such a process is shown in FIG. 1. As shown in FIG. 1, process 100 incorporates chlorination reactor 102, HCl purification unit 104, quench/drying unit 106, and separation units 108 and 110, in operation of process 100, 1,1,1,3-tetrachloropropane is provided to chlorination reactor along with aluminum chloride.

Chlorination reactor 102 produces an overhead stream comprising excess chlorine and the byproduct HCl. This overhead stream is provided to HCl purification column 104, operated at conditions effective to provide HCl as an overhead stream and a bottoms stream comprising chlorine, which can be recycled to chlorination reactor 102.

The bottom product stream from chlorination reactor is quenched and dried in drying unit 106 to remove aluminum chloride in the aqueous phase. The dried product stream from chlorination reactor 102 is provided to separation unit 108. Separation unit 108 is operated at conditions effective to provide unreacted 1,1,1,3-tetrachloropropane as an overhead stream and 1,1,1,2,3-pentachloropropane and heavier by products as a bottoms stream.

The bottoms stream from separation unit 108, comprising 1,1,1,2,3 may be provided to separation unit 110 for further purification and provision of substantially pure 1,1,1,2,3-pentachloropropane as an overhead stream therefrom.

Some embodiments of the invention will now be described in detail in the following examples.

### EXAMPLE I

### Chlorination of 1,1,1,3-tetrachloropropane to Using AlCl₃ vs. FeCl₃

In a glove box, the base of a 100 mL Parr reactor is charged with 100 mg of either FeCl₃ or AlCl₃, and methylene chloride (45 mL). The reactor is sealed, stirring is initiated (900 rpm) and the reactor is pressurized with N₂ (˜140 psig) and vented. Chlorine (30% in N₂) is passed through the reactor for 35 min at a reactor pressure of 125 psig. The shot tank is charged with 1,1,1,3-tetrachloropropane (1 mL) and methylene chloride (9 mL). Chlorine is then stopped, and the reactor is heated to 50° C. and the reactor pressure is adjusted to ˜125 psig. The shot tank is added and the reactor is sampled every two minutes for 10 minutes and then at 30 and 60 minutes. The samples are removed from the box and quenched with saturated aqueous sodium bicarbonate. The organic layer is separated. Analysis by ¹H NMR spectroscopy in deuterated chloroform indicates full conversion of 1,1,1,3-tetrachloropropane by the first sampling at 2 minutes for AlCl₃ (see Table 1). In contrast, it takes more than 1 hour to see similar conversions with FeCl₃ (see Table 2). In both Tables 1 and 2, 1113 is used as an abbreviation for 1,1,1,3-tetrachloropropane, 11123 is used as an abbreviation for 1,1,1,2,3-pentachloropropane and 111223 is used as an abbreviation for 1,1,1,2,2,3-hexachloropropane. The productivity is determined assuming a density of 1.46 g/mL for 1,1,1,3-tetrachloropropane, a reactor volume of 1.2 times the volume of the 1113 used and normalized to the % Cl₂ in the feed.

TABLE 1
Product composition (in mole %) of 1,1,1,3-tetrachloropropane
chlorination using AlCl₃ as a function of time.

| | Time (min) | | | | | | | |
|---|---|---|---|---|---|---|---|---|
| | 0 | 2 | 4 | 6 | 8 | 10 | 31.65 | 61.65 |
| 1113 | 100 | 0.00 | 0.00 | 0.00 | 0.00 | 0.00 | 0.00 | 0.00 |
| 11123 | 0 | 92.68 | 87.30 | 82.51 | 78.40 | 75.08 | 35.18 | 10.07 |
| 111223 | 0 | 7.32 | 12.70 | 17.49 | 21.60 | 24.92 | 64.82 | 89.93 |
| Conversion | | 3333.3 | 1666.67 | 1000 | 769.2 | 625 | 189.57 | |
| rate (%/hr) | | | | | | | | |
| Productivity | | 2235.3 | 1052.8 | 663.4 | 472.7 | 362.2 | 53.6 | 7.9 |
| (gr/L/min) | | | | | | | | |

TABLE 2
Product composition (in mole %) of 1,1,1,3-tetrachloropropane
chlorination using FeCl₃ as a function of time.

| | Time (min) | | | | | | | |
|---|---|---|---|---|---|---|---|---|
| | 0 | 2 | 4 | 6 | 8 | 10 | 31. | 66 |
| 1113 | 100 | 85.62 | 76.10 | 67.61 | 56.58 | 47.21 | 11.20 | 2.19 |
| 11123 | 0 | 14.38 | 23.90 | 32.05 | 43.34 | 52.03 | 87.73 | 96.83 |
| 111223 | 0 | 0.00 | 0.00 | 0.34 | 0.08 | 0.76 | 1.07 | 0.98 |
| Conversion | | 479.3 | 398.3 | 323.9 | 334 | 330 | 172.1 | 88.91 |
| rate (%/hr) | | | | | | | | |
| Productivity | | 346.8 | 288.2 | 257.7 | 261.3 | 251.0 | 136.5 | 70.8 |
| (gr/L/min) | | | | | | | | |

1. A process for the production of 1,1,1,2,3-pentachloropropane from 1,1,1,3-tetrachloropropane comprising catalyzing the chlorination of 1,1,1,3-tetrachloropropane with aluminum chloride, either alone or in combination with ferric chloride.

2. The process of claim 1, wherein aluminum chloride is used alone.

3. The process of claim for 1, wherein the process is carried out in the presence of a solvent.

4. The process of claim 3, wherein the solvent comprises carbon tetrachloride, sulfuryl chloride, one or more tetrachloropropanes, one or more pentachloropropanes, one or more hexachloropropanes, or a combination of any number of these.

5. The process of claim 1, wherein the process is carried out at a temperature of from ambient to 100° C.

6. The process of claim 1, carried out at a pressure of from ambient to 200 psig.

7. The process of claim 1, wherein at least a 80% conversion of the 1,1,1,3-tetrachloropropane is provided with productivity of greater than 360 gr/L/min.

8. The process of claim 1, wherein a yield of 1,1,1,2,3-pentachloropropane of at least 75% with productivity of greater than 360 gr/L/min.

9. The process of claim 1, wherein the 1,1,1,3-tetrachloropropane is prepared in situ.

10. The process of claim 9, wherein the 1,1,1,3-tetrachloropropane is prepared by the reaction of ethylene and carbon tetrachloride in the presence of one or more Lewis acid catalysts and optionally, a trialkylphosphate or alkylamine.

11. The process of claim 10, wherein the Lewis acid comprises ferric chloride, couprous chloride, aluminum chloride, titanium chloride, antimony pentachloride, boron trichloride, one or more lanthanum halides, one or more metal triflates, or a combination of these.

12. The process of claim 1, wherein the product stream comprising 1,1,1,2,3-pentachloropropane is dehydrochlorinated to produce 1,1,2,3-tetrachloropropene.

13. The process of claim 12, wherein the dehydrochlorination is conducted using caustic.

14. The process of claim 12, wherein the dehydrochlorination is conducted catalytically using a Lewis acid catalyst.

15. The process of claim 14, wherein the Lewis acid catalyst comprises ferric chloride, aluminum chloride, titanium chloride, antimony pentachloride, boron trichloride, one or more lanthanum halides, one or more metal triflates, or a combination of these.
